(19) United States
(12) Patent Application Publication
(10) Pub. No.: US 2007/0026130 A1
(43) Pub. Date: Feb. 1, 2007

(54) Method of developing latent fingerprints

(75) Inventor: Douglas C. Arndt, Jacksonville, FL (US)

Correspondence Address: Harold L. Jackson;Jackson Law Corporation, Ste. N, 14751 Plaza Dr., Tustin, CA, 92780, US

(73) Assignee: Armor Holdings Forensics, Inc.

(21) Appl. No.: 11/493,245

(22) Filed: Jul. 26, 2006

Related U.S. Application Data
(60) Provisional application No. 60/702,840, filed on Jul. 27, 2005.

(51) Int. Cl. A61B 5/117

(52) U.S. Cl. 427/1

(57) ABSTRACT

A method of developing latent prints deposited on a substrate e.g., paper includes impregnating a porous pad with a chemical compound in its solid state which will sublimate preferably within a temperature range of about 100° F. to 300° F. over a short time interval e.g., several minutes with the sublimated gas capable of reacting with one or more of the constituents of latent fingerprint residues to form a visible image of the fingerprint. The pad is placed in a development compartment formed in a base of the apparatus with a heating element located below the pad. The substrate bearing the latent print is placed over the pad. The compartment is closed with a lid and the compound heated to the compound sublimation temperature for a given time to allow the sublimated gas to react with the residue and the substrate is then removed.

### RELATED APPLICATIONS

This application claims priority based on U.S. Provisional Patent Application Ser. No. 60/702,840, filed Jul. 27, 2006, entitled METHOD OF DEVELOPING LATENT FINGERPRINTS. The contents of said provisional application are incorporated herein by reference.

### FIELD OF THE INVENTION

The present invention relates to the development of latent fingerprints.

### BACKGROUND OF THE INVENTION

The term “chemical developer” as used in the forensic fingerprint art refers to the visualization of the components or constituents, e.g., oils, salts, amino acids deposited by the ridge pattern of a person's fingertip, i.e., fingerprint area, onto a porous substrate such as paper. It is to be noted that the term “fingerprint” or “fingerprint area” as used herein includes palm as well as foot prints.

Several methods have been used to develop latent fingerprints particularly on porous substrates such as paper. One method, described in my U.S. Pat. No. 6,841,188 (“'188 patent”), involves the preparation of a reagent solution, i.e., 8-hydroxyquinoline or derivative by mixing it with a complexing agent such as a metal salt and applying the solution, e.g., by spraying, dipping etc. to a porous substrate wherein the solution is adsorbed and precipates onto the substrate surface allowing the precipate to highlight the latent image.

While this method has achieved considerable success it has certain disadvantages including (a) to some extent shelf life limitations, (b) possible staining or blackening of the underlying documents, (c) possible destruction of the latent print residues and (d) the requirement that the substrate be porous.

The '188 patent discusses another prior art latent print developing method which involves the reduction of a silver salt in solution to elemental silver which precipates and adsorbs onto the surface of the latent print. The disadvantages of this method are also discussed in the '188 patent.

Iodine and ruthenium tetroxide have also been used in a fuming process as well as vacuum sublimation of relatively high vapor pressure chemicals and dyes. Butane torch systems have been used to vaporize cynoacrylates and dyes. Ninhydrin and DFO solutions have also been used to develop latent prints. However, such solutions require that the materials be mixed and weighed. In addition, such solutions are generally flammable, provide inhalation hazards, often cause inks to run, and they can take several hours to weeks to develop prints. One company markets a refrigerant to which the ninhydrin or DFO can be added to overcome many of the problems, but it is quite expensive.

Inkless systems have been developed to enable a user to take the fingerprints of an individual while present as contrasted with the development of a latent fingerprint. Such inkless systems rely on the reaction of two chemical reagents at the time of fingerprint development. Typically a nonstaining first reagent (color former) such as a transition metal salt is applied to a person's fingertips and a second reagent (developer), such as 8-hydroxyquinoline or it's derivative, is pre-applied to or inherent in the recording medium such as paper. The reagents remain isolated until the fingerprint is taken. See, for example, U.S. Pat. Nos. 4,029,012; 4,182,261 and 4,262,623 (“'623 patent”). More recently the use of a chealting agent has allowed the two reagents in solution to be located in the same container by preventing the reagents from chemically reacting until exposed to the moisture from the skin and/or the recording medium. See U.S. Pat. No. 6,488,750.

The assignee of the present application and it's predecessor, one of the early pioneers in inkless/nonstaining fingerprint systems, introduced an apparatus in the late 1970's called the “Digit 10®” which is described n the '623 patent. The Digit 10® apparatus had (and as marketed today has) a reagent dispensing pad containing a color former in the form of a transition metal salt, i.e., FeCl₃ in one area and a development chamber containing a developer in the form of 8-hydroxyquinoline affixed to an open celled fibrous pad in an adjacent area. Prior to the fingerprinting procedure a paper sheet or card, preferably meeting FBI specifications, is placed in the development chamber and the finger tips, of the person to be fingerprinted, are pressed against the color former dispensing pad to coat the ridge patterns and then the fingertips are pressed against the sheet or card. The development chamber is then closed allowing the gaseous phase of 8-hydroxyquinoline to penetrate the paper and react with the FeCl₃ with the reaction product emulating the ridge patterns of the person's fingertips' ridges. The '623 patent points out that the developer chamber can be heated, e.g., to increase the sublimation rate of the developing solution. The Digit 10® apparatus provides outstanding black prints, which as required by the FBI, have an expectant 30 year lifetime.

Obviously one cannot apply a color former to the fingertips of a non-present person. I have discovered that a latent fingerprint may be quickly developed through a dry method, eliminating the need for a separate color former reagent such as FeCl₃ of the Digit 10® system while obviating the application of liquid reagents via the '188 patent system and the lengthy development time as well as the need for bulky heat sources, fuming and vacuum chambers, ventilation systems and personnel protective equipment associated with one or more of the above prior art processes. The resulting images on the substrate need not have a long life expectancy since such images may be converted to a more permanent storage medium if desired.

### SUMMARY OF THE INVENTION

A method of developing latent fingerprints on a porous, e.g., paper, or nonporous, e.g., plastic, substrate in accordance with the present invention comprises a) providing a solid chemical (developer) compound capable of sublimating sufficient quantities within a temperature range of about 72° F. and 450° F. for a short time interval so that the sublimated gaseous phase of the compound is capable of reacting with one or more of the constituents found in the residues of a latent fingerprint to form a discernable image of the fingerprint in the visible spectrum with or without exposure to external radiation and b) exposing the substrate to the chemical compound within said temperature range and for at least said short time interval to develop the image.

The later step is preferably carried out through the use of an apparatus having a development compartment with the chemical compound in gaseous communication with the compartment and means for releasably opening and closing the development compartment so that the substrate may be placed in the development compartment when open and the sublimated compound preferably may be inhibited from egressing the compartment when closed.

The substrate possessing the anticipated latent fingerprint is placed within the open development compartment which is then closed for a selected time and the compartment preferably heated to allow the sublimated chemical to react with the print residue to develop the print. The compartment is then opened and the substrate removed.

The residue constituents of latent fingerprints, of primary interest, are oils, salts and amino acids. The developer compound in a sublimated state must be capable of reacting with one or more of such constituents within a reasonably short time interval to provide either a visible image without the need for external radiation or with such radiation. Preferably the developer compound is chosen to provide fluorescence under suitable external radiation. Such external radiation may be chosen to substantially eliminate interfering background or noise fluorescence as is well known in the art.

The developer compounds that are best suited for the invention must be low-energy solids with molecular attractions (e.g., van der Waals force) keeping the solids together but which attraction is easily overcome by the application of heat.

I have found that the following developer compounds react with the listed latent fingerprint constituents at a reasonably low temperature, e.g., 72° F. to 450° F. and generally between about 100° F. to 300° F. to provide a discernable and satisfactory image of a latent fingerprint:

Amino Acids: Aldehydes, ketones, quinones (e.g. benzo-, naphtho-, anthra-), 4-(dimethylamino)cinnamaldehyde, quinolines, and dansylated compounds.

Salts: 8-hydroxyquinolines, acridine hydrochlorides

Oils: lipophylic compounds in general, and specific ones such as acridines, coumarins such as those typically used as laser dyes, low molecular weight azoles (nitrogen containing with oxygen substitution are oxazoles and nitrogen containing with sulfur substitution are thiazoles), liposomes (e.g. Nile Red), stilbenes, azos, azolines, and conjugated polycyclic aromatic compounds containing at least three fused rings that include without limitation anthracene, benzanthracene, pentacene, substituted pentacene, naphthacene, phenacene, substituted phenacene, and derivatives thereof.

Other dry developer compounds, which have been found to develop latent prints with varying degrees of effectiveness, are set forth in the following discussion of the preferred embodiment.

### BRIEF DESCRIPTION OF THE DRAWINGS

FIGS. 1, 3a and 3b are perspective views of an apparatus suitable for use in carrying out the method of the present invention with FIG. 1 showing the hinged cover in an open position exposing the heating element in the base and FIGS. 3a and 3b showing the pad carrying the chemical developer compound and the foam cushion in place on the base and cover, respectively;

FIG. 2 is a block diagram of a power supply for the heating element;

FIGS. 4a and 4b are enlarged cross-sectional views partially broken away of the cover positioned above the base and of the cover secured to the base showing the enclosed development chamber, respectively.

### DESCRIPTION OF THE PREFERRED EMBODIMENT

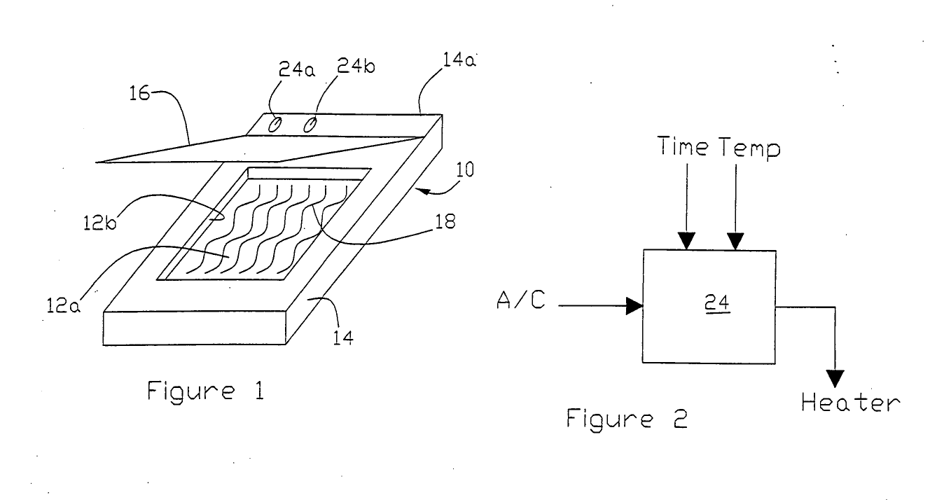

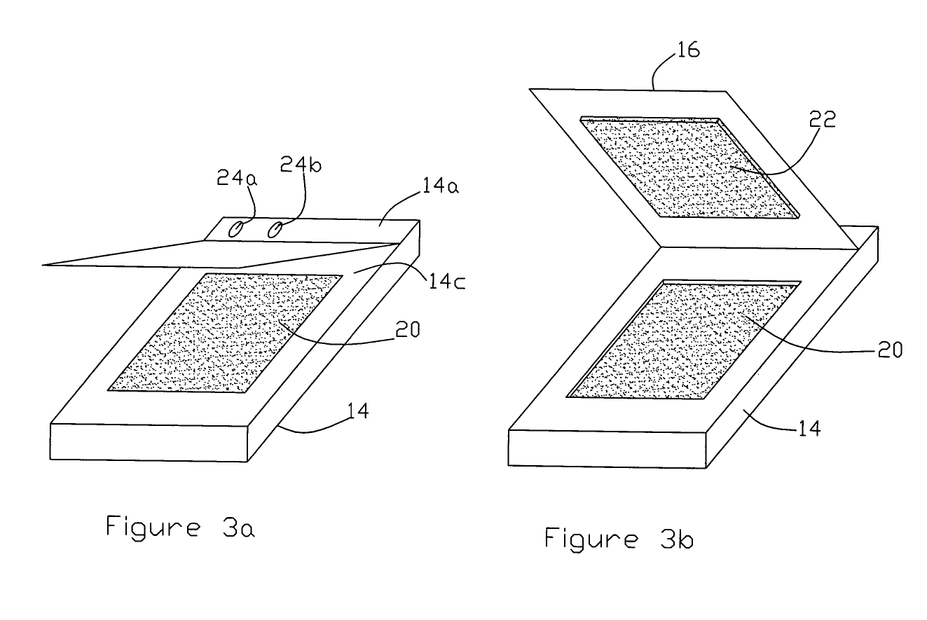

FIGS. 1-4b illustrate an apparatus 10 suitable for use in carrying out the method of the present invention. The apparatus includes a development compartment 12 formed between a central section of a base 14 and cover 16 hinged to an upper portion 14a of the base. The bottom portion 12a of the development compartment is provided with a heater in the form of electrical elements 18 positioned above the base bottom wall 11 and within an upwardly extending peripheral wall 12b terminating in a planar rim 14c surrounding the lower portion of the development compartment. A porous pad or sheet 20, such as blotter paper, containing the developer chemical compound (to be described) is disposed over the heating element. A resilient foam cushion 22 extends downwardly from the central portion of the cover and seats within the wall 12b when the cover is closed to form the development compartment within the peripheral wall and between the pad 18 and cushion 20. The development compartment may be of any convenient size. For example, a compartment capable of accommodating an 8½″×11″ sheet has been found to be quite satisfactory.

A power supply 24 is disposed within the upper section 14a of the base and is arranged to supply ac current to the heating element 18 from a standard ac outlet (not shown) at a selected temperature, via knob 24a, and for a selected time via knob 24b, the implementation of which is within the knowledge of those skilled in the art.

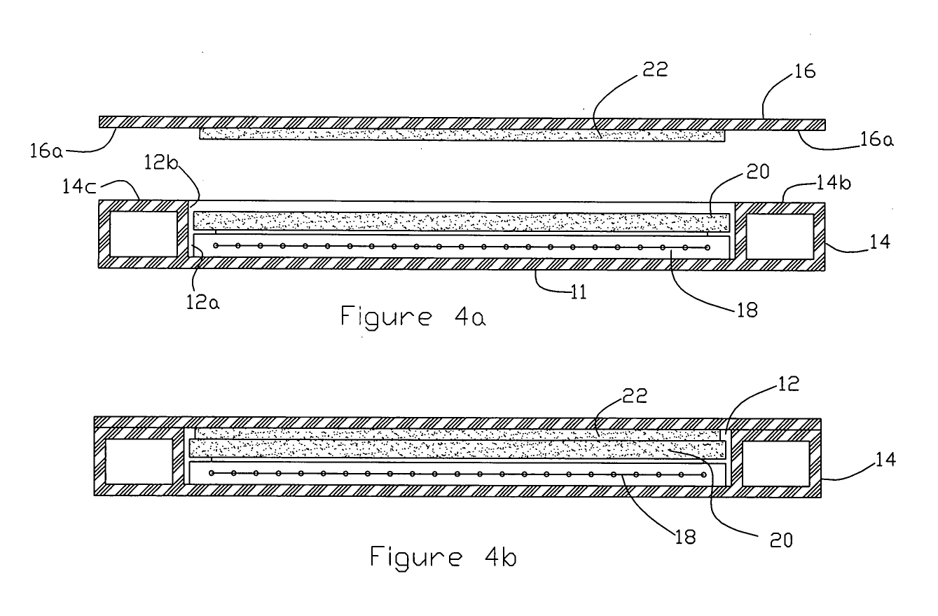

A magnetic closure may be provided between the hinged cover and base, e.g., by including a magnetic material along the border 16a and a ferrous metal border 14b on the base (around the rim 14c), to provide a seal between the cover and base to inhibit the egress of sublimated gases from the chemical compound impregnated in the pad 20 to the atmosphere. See FIG. 4a.

The following organic compounds have been found to be effective in varying degrees in carrying out the method of the present invention:

Nile Red (an oxazone dye), 7-(4-methoxybenzylamino)-4-nitrobenzozadiazole, carbazole, acridine and its derivatives, rhodamine dyes, 2-(2-hydroxyphenyl)-benzoxazole, 2-(2-hydroxyphenyl)benzothiazole, coumarin compounds such as 4-hydroxycoumarin and coumarins 1, 2, 4, 6, 7, 30, 102, 120, 138, 151, 152, 153, 307, 314, 334, 337, 338, and 343, benzotriazole, 2-chloro-mercaptobenzoxazole, 2-(2-hydroxy-5-methoxyphenyl)benzothiazole, 2-phenylbenzothiazole, 2-phenylbenzaxazole, 8-hydroxyquinoline, 8-hydroxyquinaldine, anthracene and its derivatives, naphthalene and its derivatives, 4-(dimethylamino)cinnamaldehyde, fluorescamine, phthalic dicarboxaldehyde, naphthoquinone-4-sulfonic acid, dansyl chloride and other dansylated compounds, 4-chloro-7-nitrobenzofurazan, 4-dimethyaminobenzaldehyde, 5,6-dimethylbenzimidazole, 5-chloro-2-methylbenzothiazole, chrysene, 4-hydroxybenzaldehyde, nicotinamide, and camphor.

There is some overlap between the above chemicals and those listed under the Summary of the Invention. Such compounds are capable of sublimating in sufficient quantities within a temperature range of about 100° F. to 300° F. over a short time interval, e.g., 30 seconds to several minutes, with the sublimated gaseous phase of the compound capable of reacting with one or more of the constituents found in the residues of latent fingerprints to form a discernable colorant or fluorescent representation of the print. The term “short time” as used herein is not to be considered limiting in the sense that an exposure time of say one hour or possibly more would be beyond the scope of the invention. Of the above, the preferred chemical compounds at the present time are Nile Red and 4-(dimethylamino)cinnamaldehyde. However, additional investigation will undoubtedly expand this list.

In addition I believe that the following compounds would also function as dry developer reagents to develop latent fingerprints in accordance with this invention:

Anthranilic acid, 1,4-naphthoquinone, benzanthrone, tetracene, pentacene, 2-(2-hydroxy-4-methylphenyl)-4-(3)-quinazolone, 2-(3,5-dichloro-2-hydroxyphenyl)-4-(3)-quinazolone, 2-(5-chloro-2-hydroxyphenyl)-4-(3)-quinazolone, 2-(2-hydroxy-3-methylphenyl)-4-(3)-quinazolone, 2-(4-ethyl-2-hydroxyphenyl)-4-(3)-quinazolone, anthraquinone, 1,4-benzoquinone, and salicylic acid.

The pad 20 is readily prepared by dissolving the chosen developer compound, preferably substantially pure, in a volatile organic solvent such as acetone or perhaps ethyl alcohol and simply soaking the pad 20 with the solution and allowing the solvent to evaporate. The evaporated solvent leaves behind finely dispersed crystals (or powder) within the fibrous structure of the pad.

In the alternative, the pad may be in the form of a pouch open at one end with the chosen developer compound in crystalene or power form uniformly dispersed within the pouch without the use of a solvent.

To perform the method of my invention, the pad 20, so prepared, is placed in the lower section of the development compartment 12, i.e., above the heating element 18 of the above described (or equivalent) apparatus.

A porous substrate, e.g., paper, in the form of a check or other document, or a nonporous substrate, e.g., plastic disc or sheet bearing an anticipated latent fingerprint is placed within the open development compartment. The cover 16 is then closed, sandwiching the substrate (preferably with the latent fingerprint specimen facing the developer pad) between the foam cushion 22 and the pad 20. The temperature and exposure time are then set (unless set at the factory) via the controls 24a and 24b to allow the sublimated gas to develop the latent print. The temperature setting will be dependent upon the developer compound used. For example, I have found that a temperature of about 140° F. is sufficient to sublimate sufficient quantities of 4-(dimethylamino)cinnamaldehyde to react with the amino acids inherent in latent print residues to provide a highly fluorescent image of the latent print in about 30 seconds of exposure time. This chemical reaction also suppresses interfering background fluorescence common with some of the prior art latent print developing methods. Nile Red requires a higher setting, e.g., about 220° F. to selectively bind to the oily components inherent in the sebaceous residues to provide a fluorescent image. Fluorescent developer reagents generally provide higher sensitivity than that of common, colorimetric reagents. The dry process in general also has these advantages:

1. The reagent is evenly distributed over the document

2. There is no need for solvents, which may destroy the document or the latent prints, produce a fire or an inhalation hazard, run inks, and require time to dry

3. In the vapor phase, the reactive “particles” are molecular in size and therefore able to achieve much greater resolution

4. The process is rapid (seconds compared to, in some cases, weeks)

5. No mixing of chemicals

6. No shelf-life limitations due to chemical instabilities

7. Inexpensive in comparison chemical solutions

8. No hazardous waste to dispose of and it can be used sequentially with the traditional methods in widespread use today. It should be noted that the developer reagent dispensing pad, properly prepared, will accommodate the development of many latent prints, e.g., 50-100.

There has been described a novel and highly useful method of developing latent fingerprints. Various modifications are possible within the generic principles disclosed by this invention.

What is claimed is:

1. A method of developing latent fingerprints deposited on a substrate comprising:
a) providing a solid chemical compound capable of sublimating in sufficient quantities within a temperature range of about 72° F. to 450° F. over a short time interval with the sublimated gaseous phase of the compound capable of reacting with one or more of the constituents found in the residues of latent fingerprints to form a discernable colorant or fluorescent representation of the fingerprint;
b) providing an apparatus having a development compartment with the chemical compound in fluid communication with the compartment and means for releasably opening and closing the development compartment so that the substrate may be placed in the development compartment when open and the sublimated compound preferably may be inhibited from egressing the compartment when closed;
c) opening the compartment and placing the substrate with a latent fingerprint thereon and closing the compartment;
d) heating the compartment to a temperature within said range;
e) leaving the compartment closed for at least said short time interval; and
f) opening the compartment and removing the substrate therefrom.

2. The method of claim 1 wherein the compartment is provided with a heater and actuating the heater to heat the compound during the development stage.

3. The method of claim 1 wherein the chemical compound is selected from one or more of the group consisting of:
Aldehydes, ketones, quinones (e.g. benzo-, naphtho-, anthra-), 4-(dimethylamino)cinnamaldehyde, quinolines, and dansylated compounds, 8-hydroxyquinolines, acridine hydrochlorides, lipophylic compounds including acridines, coumarins, low molecular weight azoles, liposomes (e.g. Nile Red), stilbenes, azos, azolines, and conjugated polycyclic aromatic compounds containing at least three fused rings that include without limitation anthracene, benzanthracene, pentacene, substituted pentacene, naphthacene, phenacene, substituted phenacene, and derivatives thereof.

4. The method of claim 1 wherein the chemical compound is selected from one or more of the group consisting of:
Nile Red (an oxazone dye), 7-(4-methoxybenzylamino)-4-nitrobenzozadiazole, carbazole, acridine and its derivatives, rhodamine dyes, 2-(2-hydroxyphenyl)-benzoxazole, 2-(2-hydroxyphenyl)benzothiazole, coumarin compounds including 4-hydroxycoumarin and coumarins 1, 2, 4, 6, 7, 30, 102, 120, 138, 151, 152, 153, 307, 314, 334, 337, 338, and 343, benzotriazole, 2-chloro-mercaptobenzoxazole, 2-(2-hydroxy-5-methoxyphenyl)benzothiazole, 2-phenylbenzothiazole, 2-phenylbenzaxazole, 8-hydroxyquinoline, 8-hydroxyquinaldine, anthracene and its derivatives, naphthalene and its derivatives, 4-(dimethylamino)cinnamaldehyde, fluorescamine, phthalic dicarboxaldehyde, naphthoquinone-4-sulfonic acid, dansyl chloride and other dansylated compounds, 4-chloro-7-nitrobenzofurazan, 4-dimethyaminobenzaldehyde, 5,6-dimethylbenzimidazole, 5-chloro-2-methylbenzothiazole, chrysene, 4-hydroxybenzaldehyde, nicotinamide, and camphor.

5. The method of claim 1 wherein the chemical compound is selected from one or more of the group consisting of:
Anthranilic acid, 1,4-naphthoquinone, benzanthrone, tetracene, pentacene, 2-(2-hydroxy-4-methylphenyl)-4-(3)-quinazolone, 2-(3,5-dichloro-2-hydroxyphenyl)-4-(3)-quinazolone, 2-(5-chloro-2-hydroxyphenyl)-4-(3)-quinazolone, 2-(2-hydroxy-3-methylphenyl)-4-(3)-quinazolone, 2-(4-ethyl-2-hydroxyphenyl)-4-(3)-quinazolone, anthraquinone, 1,4-benzoquinone, and salicylic acid.

6. The method of claim 2 wherein the compartment is heated to a temperature within the range of about 100° F. to 300° F.

7. A method of developing a latent fingerprint on a substrate comprising:
a) providing a solid chemical compound capable of sublimating sufficient quantities within a temperature range of about 72° F. and 450° F. for a short time interval so that the sublimated gaseous phase of the compound is capable of reacting with one or more of the constituents found in the residues of a latent fingerprint to form a discernable image of the fingerprint in the visible spectrum with or without exposure to external radiation; and
b) exposing the substrate to the chemical compound within said temperature range and for at least said short time interval to develop the image.

8. The method of claim 7 wherein the compound is selected from one or more of the group consisting of:
Aldehydes, ketones, quinones (e.g. benzo-, naphtho-, anthra-), 4-(dimethylamino)cinnamaldehyde, quinolines, and dansylated compounds, 8-hydroxyquinolines, acridine hydrochlorides, lipophylic compounds including acridines, coumarins, low molecular weight azoles, liposomes (e.g. Nile Red), stilbenes, azos, azolines, and conjugated polycyclic aromatic compounds containing at least three fused rings that include without limitation anthracene, benzanthracene, pentacene, substituted pentacene, naphthacene, phenacene, substituted phenacene, and derivatives thereof.

9. The method of claim 7 wherein the compound is selected from one or more of the group consisting of:
Nile Red (an oxazone dye), 7-(4-methoxybenzylamino)-4-nitrobenzozadiazole, carbazole, acridine and its derivatives, rhodamine dyes, 2-(2-hydroxyphenyl)-benzoxazole, 2-(2-hydroxyphenyl)benzothiazole, coumarin compounds including 4-hydroxycoumarin and coumarins 1, 2, 4, 6, 7, 30, 102, 120, 138, 151, 152, 153, 307, 314, 334, 337, 338, and 343, benzotriazole, 2-chloro-mercaptobenzoxazole, 2-(2-hydroxy-5-methoxyphenyl)benzothiazole, 2-phenylbenzothiazole, 2-phenylbenzaxazole, 8-hydroxyquinoline, 8-hydroxyquinaldine, anthracene and its derivatives, naphthalene and its derivatives, 4-(dimethylamino)cinnamaldehyde, fluorescamine, phthalic dicarboxaldehyde, naphthoquinone-4-sulfonic acid, dansyl chloride and other dansylated compounds, 4-chloro-7-nitrobenzofurazan, 4-dimethyaminobenzaldehyde, 5,6-dimethylbenzimidazole, 5-chloro-2-methylbenzothiazole, chrysene, 4-hydroxybenzaldehyde, nicotinamide, and camphor.

10. The method of claim 7 wherein the compound is selected from one or more of the group consisting of:
Anthranilic acid, 1,4-naphthoquinone, benzanthrone, tetracene, pentacene, 2-(2-hydroxy-4-methylphenyl)-4-(3)-quinazolone, 2-(3,5-dichloro-2-hydroxyphenyl)-4-(3)-quinazolone, 2-(5-chloro-2-hydroxyphenyl)-4-(3)-quinazolone, 2-(2-hydroxy-3-methylphenyl)-4-(3)-quinazolone, 2-(4-ethyl-2-hydroxyphenyl)-4-(3)-quinazolone, anthraquinone, 1,4-benzoquinone, and salicylic acid.
